(19) United States
(12) Patent Application Publication
(10) Pub. No.: US 2011/0144534 A1
(43) Pub. Date: Jun. 16, 2011

(54) CELL COLLECTION AND TRANSFER DEVICE AND METHODS OF USE

(75) Inventor: Peter GOMBRICH, Salinas, CA (US)

(73) Assignee: CerMed Corporation, Monterey, CA (US)

(21) Appl. No.: 12/635,638

(22) Filed: Dec. 10, 2009

(51) Int. Cl. A61B 10/02

(52) U.S. Cl. 600/572

(57) ABSTRACT

A cell sample collection and transfer device is provided. The device may include a plunger configured to slide to a plurality of positions for controlling an expandable tip. The expandable tip may be operable to collect cell samples from a target site. In one example, the expandable tip may be further operable to transfer the collected cell samples when placed in contact with a receiving surface. In another example, the expandable tip may be removed from the device and placed in a storage container for further processing.

### FIELD

The present application relates generally to collecting and transferring cell samples and devices for performing the same.

### RELATED ART

It is often necessary to collect and test cell samples from patients for the purpose of detecting a number of diseases and abnormalities. For example, exfoliated cells of the uterine cervix may be collected and analyzed by cytotechnicians and pathologists to detect cervical cancer. Early detection and treatment of such a condition greatly increases the chance the patient will be cured.

As a result, many devices have been developed to assist health care providers to collect and transfer the desired cell samples from patients. For example, U.S. Pat. No. 6,663,576 describes a device for the collection of cervical cells for the purpose of screening for cervical cancer. The collected cells may be analyzed directly on the collection and transfer device, or alternatively, the collected cells may be removed from the collection and transfer device using a liquid solution and subsequently transferred to a slide. However, one drawback is that the device does not allow the user to transfer the collected cells to a receiving surface directly from the collection and transfer device.

Another cell collection and transfer device is described by U.S. Patent Application Publications Number 2009/0105610 and 2009/0017474. The device allows the user to extend an expandable tip made of elastomeric material to contact and collect cell clusters. Once the cell clusters have been collected by the expandable tip, the device may be attached to a receiving structure to transfer the cells from the expandable tip to a second surface, for example, a slide or Petri dish. In one example, the expandable tip may be inflated prior to transfer of the cells. One drawback of the described device is that the operation of a mechanically expandable tip is complex and requires greater operator skill. Another drawback is that the device is intended for use with a separate apparatus for accurately transferring the cell samples to a receiving surface.

Therefore, it would be desirable to have a self-contained and easy-to-use device that is capable of collecting cell samples and accurately transferring them to a receiving surface while preserving the spatial relationship between the cell samples without the use of any additional apparatus. It would also be desirable for the device to include a collector operable to conform to the target site to increase the surface area of the collector placed in contact with the target site.

### BRIEF SUMMARY

An apparatus for collecting and transferring cell samples, the apparatus having a main body, an expandable tip coupled to the main body that is configured to inflate to a first inflation level for collecting a cell sample, inflate to a second inflation level for transferring at least a portion of the collected cell sample, and deflate, at a rate of deflation, to a deflated level when the cell sample is being transferred. The apparatus further includes a valve, for controlling the rate of deflation of the expandable tip; and a plunger disposed within the main body configured to generate positive pressure in the expandable tip.

A method of using an apparatus, as described herein, for collecting and transferring cells, the method including pulling a vacuum to collapse the tip of the apparatus to allow for visualization of the endo-cervical canal; inserting the apparatus into the endo-cervical canal and contacting the cervix; putting air into the tip to expand the tip; before removing device, rotating the device 20 degrees to collect cells; removing the device from the endo-cervical canal; putting additional air into tip to expand the tip further; applying pressure to the tip to relieve pressure at a controlled rate allowing for transfer of the collected cells.

### BRIEF DESCRIPTION OF THE DRAWINGS

FIG. 1 illustrates an exemplary cell collector.

FIGS. 2A and 2B illustrate the operation of an exemplary cell collector.

FIG. 3 illustrates the operation of an exemplary cell collector.

FIG. 4 illustrates an exploded view of an exemplary cell collector.

FIG. 5 illustrates the operation of an exemplary cell collector.

FIG. 6 illustrates the operation of an exemplary cell collector.

FIG. 7 illustrates the operation of an exemplary cell collector.

FIG. 8 illustrates the operation of an exemplary cell collector.

FIG. 9 illustrates a front view and side view of the tip of an exemplary cell collector in two different configurations.

FIG. 10 illustrates the operation of an exemplary cell collector.

FIG. 11 illustrates the operation of an exemplary cell collector.

FIG. 12 illustrates the operation of an exemplary cell collector.

FIG. 13 illustrates an exemplary cell collector transferring a cell sample onto a receiving surface.

FIG. 14 illustrates another view of an exemplary cell collector transferring a cell sample onto a receiving surface.

FIG. 15 illustrates the tip of an exemplary cell collector being placed in a vial for further processing.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Various embodiments are described below relating to a cell collection and transfer device and methods for its use. The cell collection and transfer device may include a slotted plunger configured to slide to a plurality of positions for controlling an expandable tip. The expandable tip may be made of a polymer operable to collect cell samples when put in contact with a target site. The expandable tip may be further operable to transfer the collected cell samples when put in contact with a receiving surface, such as a slide, Petri dish, or the like. By collecting and transferring the cell samples in such a manner, the spatial or topological relationships between the collected cells are preserved. This allows a cytotechnician, for example, to extract additional information from the samples that would otherwise be lost using a conventional sampling method.

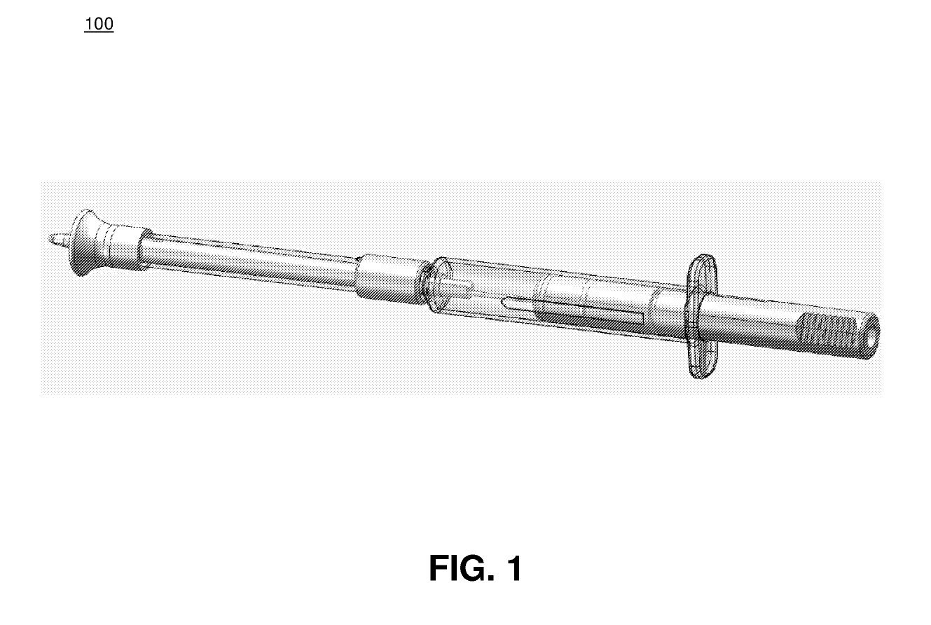

FIG. 1 illustrates an exemplary view of collection and transfer device 100 used for the collection of cell samples. For purposes of explanation, collection and transfer device 100 will be discussed with respect to the collection of cells from a cervix to screen for cervical cancer. However, it will be appreciated that collection and transfer device 100 may be used to collect cells from other regions of the body for other purposes. For example, cells may be collected from the bladder to screen for bladder cancer, the lungs to screen for lung cancer, the colon to screen for colon cancer, etc.

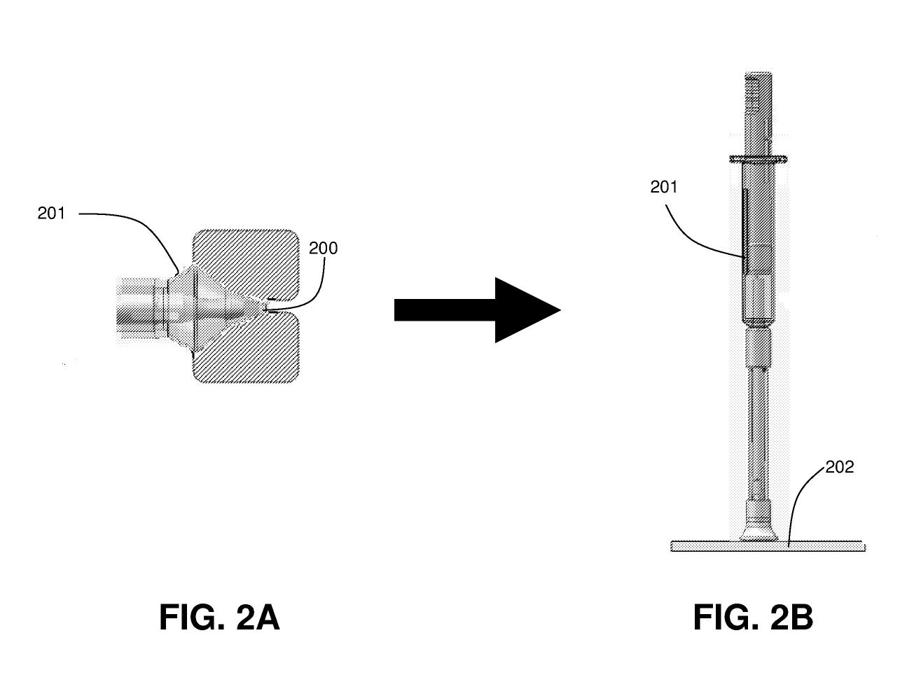

FIGS. 2A and 2B illustrate the concepts of an exemplary use of collection and transfer device 100 in accordance with embodiments of the present application. In one example illustrated by FIG. 2A, expandable tip 201 of collection and transfer device 100 is inserted into cervical canal 200 for the collection of cell samples. Expandable tip 201 is formed of a material having properties such that cells adhere to the surface of expandable tip 201 when placed in contact with the surface. Once the cell samples are collected on the surface of expandable tip 201, the cells are transferred to a receiving surface 202 by pressing or stamping the expandable tip on the receiving surface, as illustrated by FIG. 2B. Receiving surface 202 may be any surface that the user may want to transfer cell samples to, such as a slide, Petri dish, and the like.

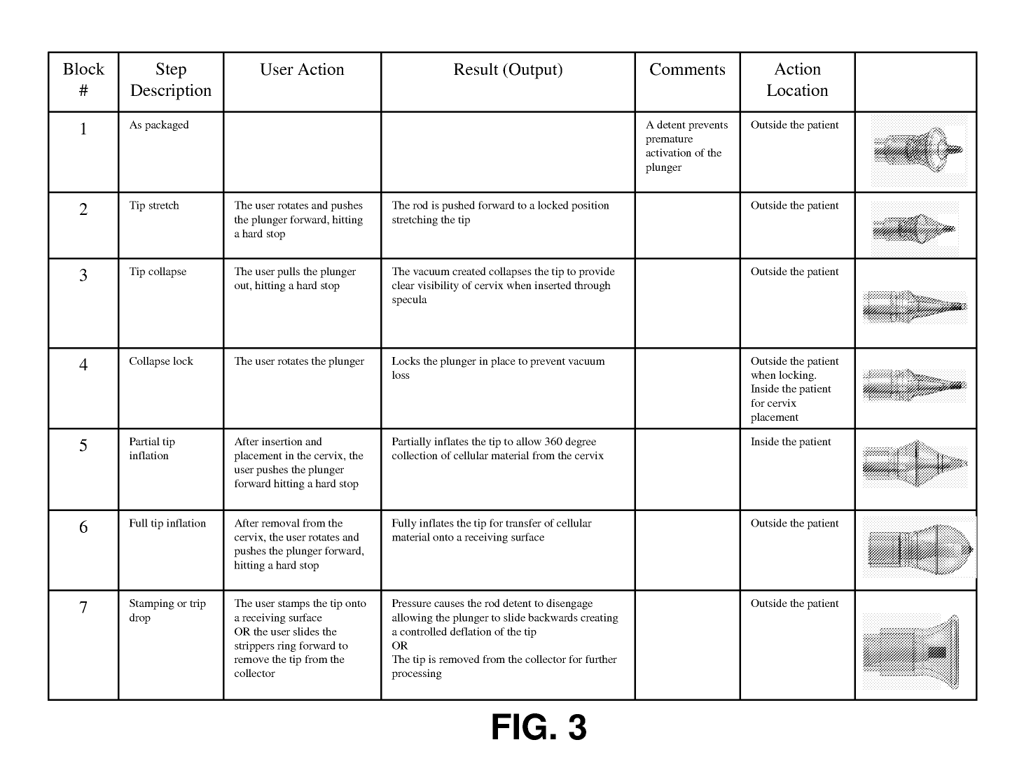

For the device described with respect to FIGS. 2A and 2B, it is desirable for the device to allow for easy insertion into the cervix, provide clear visibility of the target site during insertion, and preserve the spatial relationship between cells while collecting and transferring cell samples. To accomplish these goals, one or more of the blocks illustrated by FIG. 3 is performed. The blocks of FIG. 3 show an example using a pin inserted into a slotted plunger for controlling the stages of the collection and transfer device. It should be appreciated that other implementations may be used to achieve the goals recited above.

At block 1 of FIG. 3, collection and transfer device 100 is in an idle state, for example, while packaged for distribution. A detent at the starting position prevents the plunger of collection and transfer device 100 from prematurely activating. In one embodiment, the device is sterilized prior to packaging.

At block 2 of FIG. 3, the user may prepare the device for operation by rotating the plunger from the starting position and pushing the plunger forward until hitting a hard stop. As a result, a push rod extends forward stretching the tip of collection and transfer device 100. When the hard stop is reached, the push rod is locked in place.

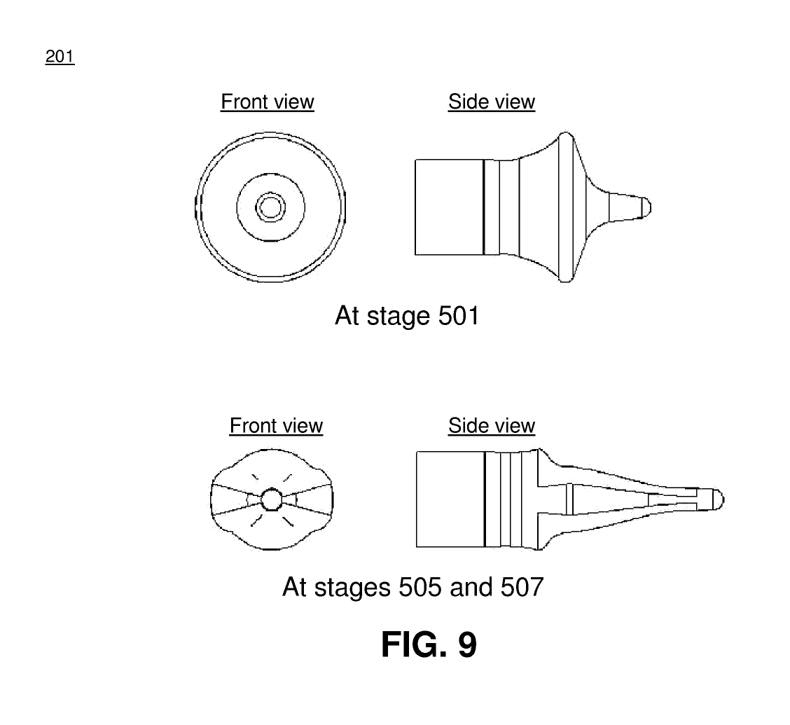

At block 3 of FIG. 3, the user may pull back on the plunger until reaching a hard stop. However, the push rod does not retract, as it is in a locked position. This creates a vacuum in the housing of collection and transfer device 100 causing the tip to at least partially collapse. The reduced tip profile allows the user improved visibility of the target site and insertion of collection and transfer device 100 through the scapula. In some embodiments, as discussed in more detail below with reference to FIG. 9, the tip may collapse in a non-uniform manner to assist in visualization of the cervix.

At block 4 of FIG. 3, the user may rotate the plunger to a locked position. This may prevent the plunger from sliding forward due to the negative pressure within the housing of collection and transfer device 100. It should be appreciated that the operations performed at blocks 1-4 are for preparing the device for use in a patient. In other examples, these blocks may be omitted and the device may instead be packaged and distributed as shown at block 4. Once the plunger has been locked in place, the device is inserted into the patient and positioned against the cervix.

At block 5 of FIG. 3, the user may push the plunger forward until hitting a hard stop, thereby partially inflating the tip of collection and transfer device 100. This allows the tip of collection and transfer device 100 to make 360 degree contact with the cervix and creates a larger surface area for collecting cellular material. By using contact sampling, the spatial orientation of the cells is preserved. One advantage of preserving the spatial orientation is that abnormal cells are more easily identified because the abnormal cells are concentrated in clusters rather than dispersed into a much larger group of normal cells. This allows the cytotechnician to look for cervical lesions rather than individual abnormal cells. Another advantage of preserving the spatial orientation of cell samples is that the location of the abnormal cells on the cervix is determined by mapping the location of the abnormal cells in the cell sample onto the cervix. Once the cell samples have been gathered on the surface of the tip of collection and transfer device 100, the user may remove the device from the patient.

At block 6 of FIG. 3, the user may rotate and push the plunger forward until hitting a hard stop. This causes the tip of collection and transfer device 100 to fully inflate for transferring the cell samples to a receiving surface.

At block 7 of FIG. 3, the user may either stamp the cell samples onto a receiving surface or remove and store the tip for future processing. To stamp the cell samples onto a receiving surface, the user may press the tip of collection and transfer device 100 down onto a receiving surface (e.g., a slide or Petri dish). The pressure applied to the tip of collection and transfer device 100 causes the push rod lock to disengage, thereby allowing the rod and plunger to slide backwards and deflate the tip. The tip of collection and transfer device 100 then flattens out and matches the contours of the receiving surface. By flattening out, the cell samples are transferred in a single motion while maintaining their spatial relationships. In one example, the stamping is done by hand, without the use of any additional machinery or apparatus. The user may remove the tip from collection and transfer device 100 by sliding the stripper ring forward and pushing the tip off of the device. The tip may then be placed in a container for storing for later processing.

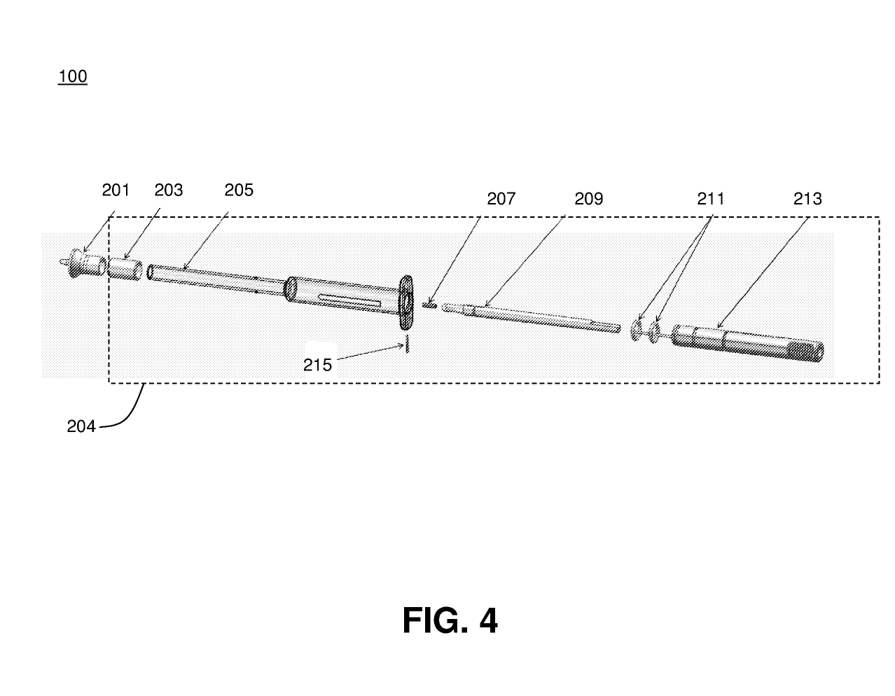

Each of the blocks presented above will be described in greater detail with respect to FIGS. 4-15.

FIG. 4 illustrates an exemplary exploded view of collection and transfer device 100. Collection and transfer device 100 may include expandable tip 201 made of an elastic polymer operable to collect cell samples on its surface when put in contact with a test region containing cell samples. Additionally, expandable tip 201 is further operable to transfer the collected cell samples when put in contact with a receiving structure, such as a slide, Petri dish, or the like. Thus, expandable tip 201 is used to collect and transfer cell samples by first placing the tip in contact with a target site containing the desired cell samples and subsequently transferring the cell samples to a receiving surface by “stamping” the expandable tip 201 onto the receiving surface.

In another example, expandable tip 201 is operable to transfer only a portion of the cell samples contained on the surface of expandable tip 201 when put in contact with a receiving surface. This may allow the user of collection and transfer device 100 to stamp the cell samples onto multiple surfaces. For example, the user may take a single sample of cells using collection and transfer device 100 and stamp the sample onto two different slides, thereby creating two slides for analysis from a single sample. In one example, expandable tip 201 is operable to transfer at least 50,000 cell samples on a first transfer to a receiving surface. Expandable tip 201 is further operable to transfer at least an additional 5,000 cell samples on a second transfer to a receiving surface.

Expandable tip 201 may be made of any elastomeric material, such as microporous polyvinyl acetate, nitrile rubber, silicone rubber, latex rubber, polyurethane, and the like. The material selected to create expandable tip 201 may affect various operational aspects of collection and transfer device 100, such as expansion properties of expandable tip 201, the number of cell samples collected, the number of cell samples transferred on a first stamp, and the number of cell samples transferred on subsequent stamps. In one example, a material operable to collect at least 55,000 cell samples, transfer at least 50,000 cell samples on a first stamp, and transfer at least 5000 cell samples on a second stamp is selected. In one example, expandable tip 201 is formed using Dynaflex™ G6713-0001.

Collection and transfer device 100 may further include main body 204 connected to expandable tip 201. In one example, main body 204 may include housing 205 having a shape similar to that of a conventional syringe. For instance, housing 205 may have two hollow cylindrical portions of different diameters. Housing 205 may further include an open end connected to expandable tip 201. In one example, expandable tip 201 is secured around the outer surface of housing 205 forming an airtight seal.

In one example, main body 204 may further include stripper ring 203 for removing expandable tip 201 from housing 205. Stripper ring 203 is formed of any hard material, such as plastic, metal, or the like. Stripper ring 203 is placed around the portion of housing 205 connected to expandable tip 201. A user may slide stripper ring 203 along the surface of housing 205 to force expandable tip 201 from housing 205 without having to contact expandable tip 201, thus preventing contamination of the tip's surface.

Main body 204 may further include extension tip 207 for projecting into and extending expandable tip 201. The operation of extending expandable tip 201 with extension tip 207 will be discussed in greater detail below. Extension tip 207 is made from various materials such as rubber, plastic, metal, and the like. Main body 204 may further include push rod 209 for pushing extension tip 207 into expandable tip 201. Push rod 209 is made of any material capable of withstanding a compressive force, such as plastic, metal, and the like. In one example, extension tip 207 is connected to an end of push rod 209.

Main body 204 may further include plunger 213 for pushing push rod 209 and forcing air through housing 205 to inflate/deflate expandable tip 201. In one example, plunger 213 may include an extension portion to contact push rod 209. This extension allows additional space to be created between push rod 209 and plunger 213. In another example, plunger 213 may not be connected to push rod 209. Instead, plunger 213 is configured to make contact with push rod 209 when plunger 213 is pushed in. This allows plunger 213 to advance push rod 209 when plunger 213 is pushed in while not retracting push rod 209 when plunger 213 is pulled out.

In one example, the inner diameter of one of the hollow portions of housing 205 is similar to the diameter of plunger 213. Thus, when plunger 213 is inserted in to housing 205, an airtight seal is formed between the inner surface of housing 205 and the outer surface of plunger 213. O-rings 211 may also be placed along the surface of plunger 213 to create a better seal between housing 205 and plunger 213. In another example, the inner diameter of the second hollow portion of housing 205 is larger than the diameter of push rod 209. This configuration allows air to pass through the second hollow portion of housing 205 into expandable tip 201 when push rod 209 is inserted.

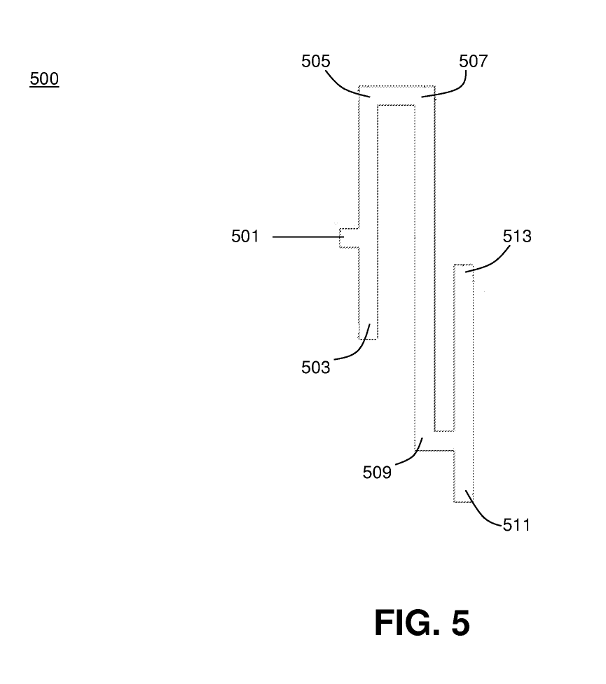

In another example, one or more slots are carved into the outer surface of plunger 213. The one or more slots are configured to receive guide pin 205. When plunger 213 is inserted into housing 205, guide pin 215 are inserted through a portion of housing 205 and positioned such that the end of guide pin 215 falls within the one or more slots on the outer surface of plunger 213. By placing guide pin 215 in the one or more slots, the movement of plunger 213 within housing 205 is restricted to motions which allow guide pin 215 to traverse the contours of the one or more slots. An example of a slotted pattern that may be placed on the surface of plunger 213 is illustrated by FIG. 5. Slotted pattern 500 includes 7 different stages 501, 503, 505, 507, 509, 511, and 513 corresponding to a different position of plunger 213 in the operation of collection and transfer device 100. While slotted pattern 500 includes 7 stages, it will be appreciated that any pattern having any number of stages may be used. In another example, the slotted pattern may alternatively be placed on housing 205 while guide pin 215 is connected to plunger 213.

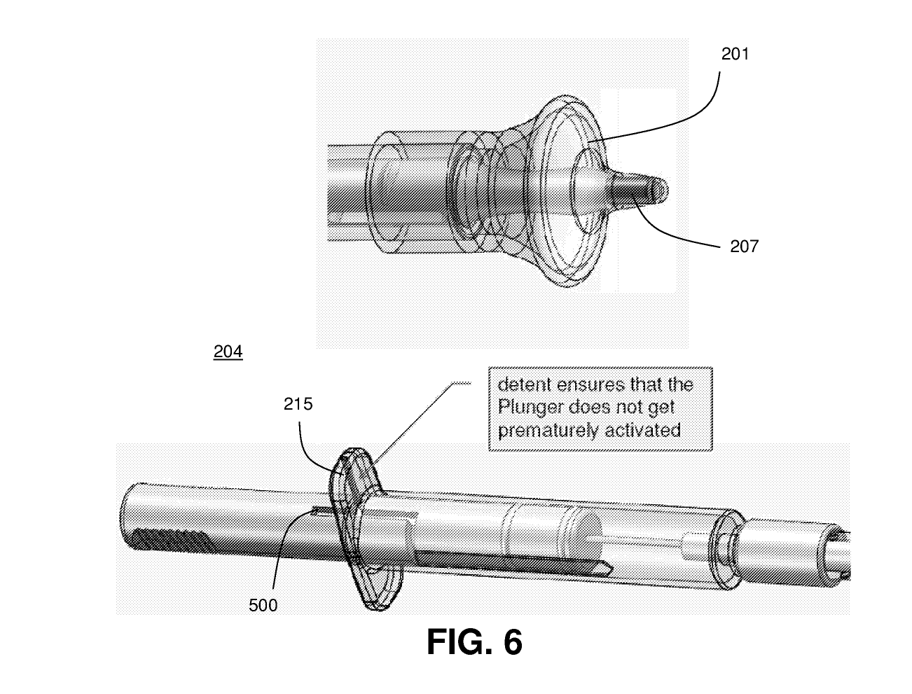

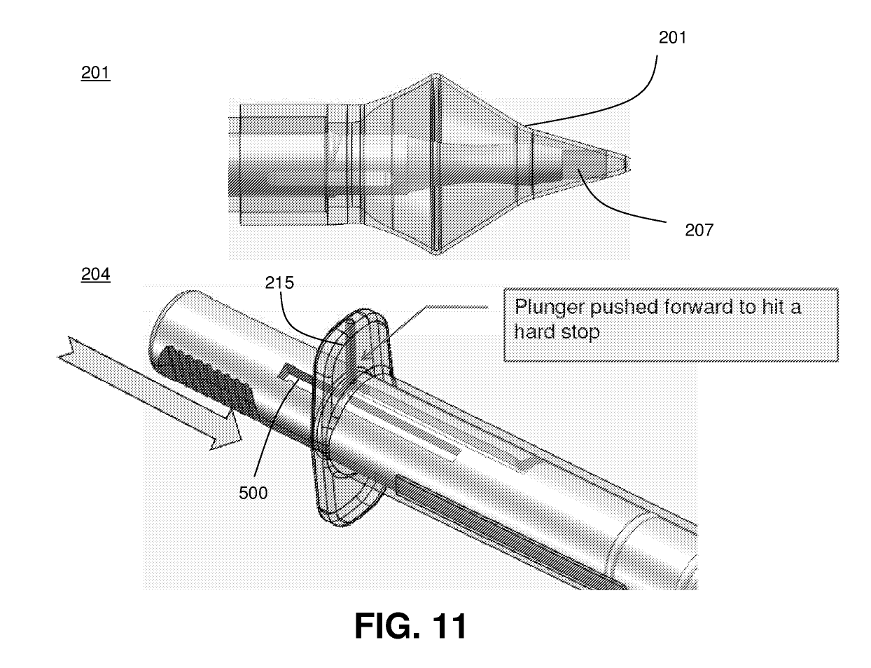

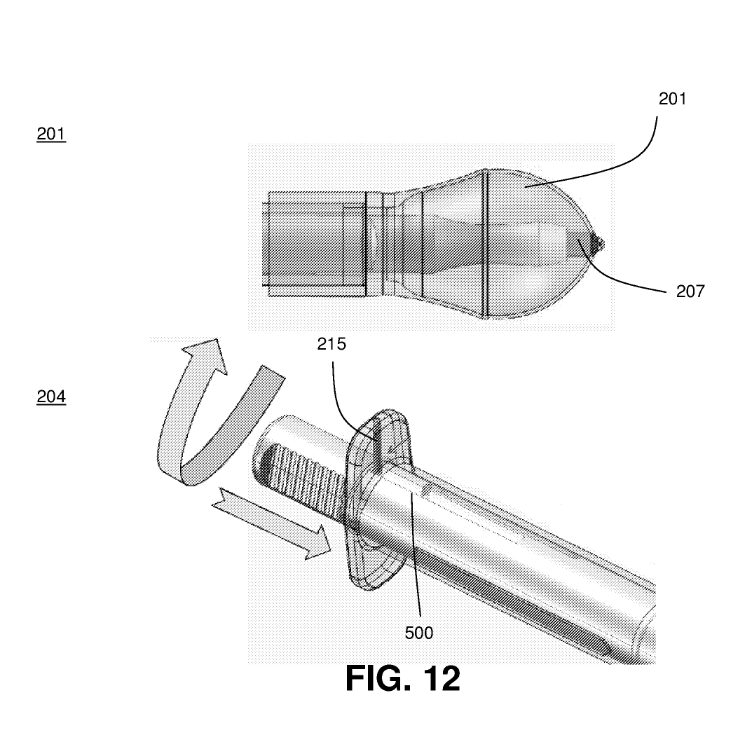

FIGS. 6-15 illustrate an exemplary operation for collecting and transferring cell samples using collection and transfer device 100. FIGS. 6-10 illustrate an exemplary process for preparing collection and transfer device 100 for collecting cell samples that is performed while the device is outside of the patient's body. FIG. 11 illustrates an exemplary process for collecting cell samples and is performed while collection and transfer device 100 is inside the patient's body. FIGS. 12-15 illustrate an exemplary process for transferring the collected cell samples from expandable tip 100 to another surface that is performed while the device is outside of the patient's body.

FIG. 6 illustrates a view of expandable tip 201 and the corresponding view of main body 204 while the device is in an idle configuration. In one example, this configuration may represent the state of collection and transfer device 100 described above with respect to block 1 of FIG. 3. As can be seen in the lower image of FIG. 6, plunger 213 is positioned such that guide pin 215 is located at stage 501 of slotted pattern 500. The corresponding view of expandable tip 201 shows that when guide pin 215 is at stage 501, expandable tip 201 is in an un-extended state. In one example, a detent is placed at stage 501 to prevent plunger 213 from sliding and prematurely activating.

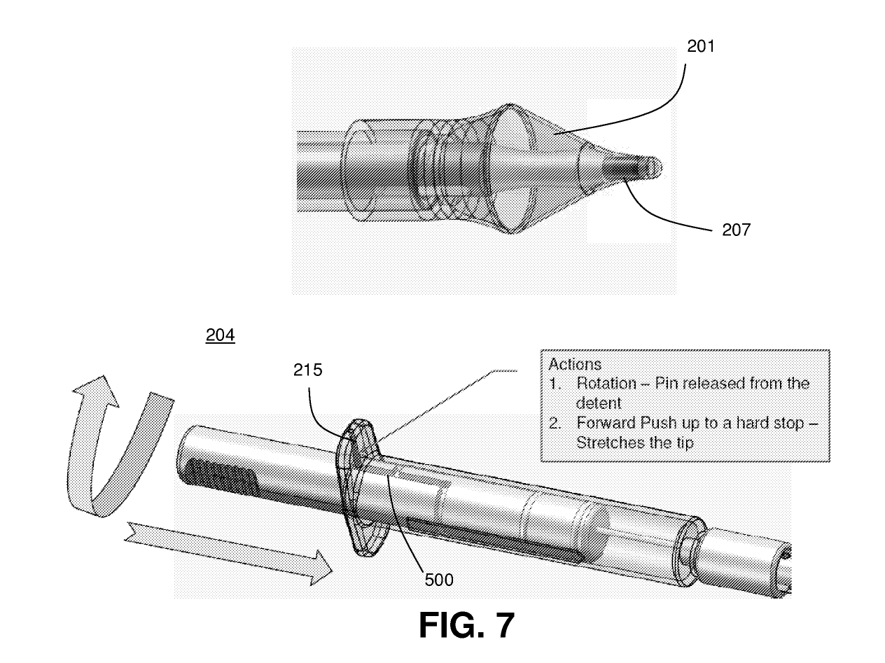

FIG. 7 illustrates an exemplary process for extending expandable tip 201 for preparing the device for collection. This process corresponds with block 2 of FIG. 3 described above. To extend expandable tip 201, the user may rotate plunger 213 counter-clockwise as illustrated by FIG. 7 to move guide pin 215 from the detent of stage 501. Once out of the detent of stage 501, plunger 213 may then be pushed in until reaching stage 503 of slotted pattern 500. As plunger 213 is pushed in, push rod 209 is also forced forward pushing extension tip 207 into expandable tip 201. The pressure from extension tip 207 causes expandable tip 201 to stretch and project forward. FIG. 7 illustrates expandable tip 201 and a corresponding view of main body 204 after the user has extended expandable tip 201. In one example, a locking mechanism (not shown) may hold push rod 209 in place once plunger 213 is pushed to stage 503. This locking mechanism may prevent push rod 209 and extension tip 207 from retracting when plunger 213 is pulled back. In one example, the locking mechanism is a detent that prevents push rod 209 from retracting after being advanced forward past the detent. In another example, the locking mechanism is a rotational lock that requires the user to push and rotate push rod 209 in to a locked position.

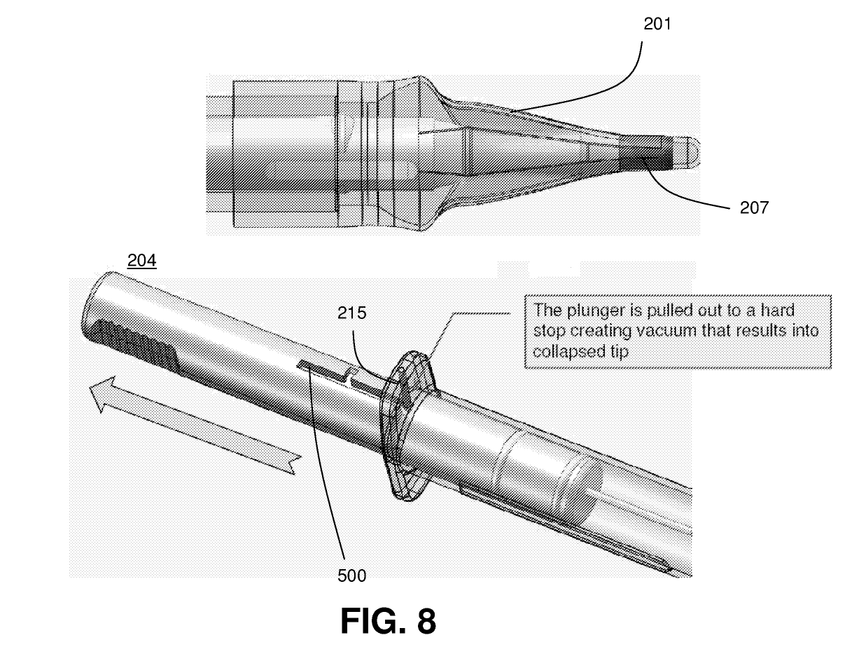

FIG. 8 illustrates an exemplary process for collapsing expandable tip 201 for insertion into the patient. This process corresponds with block 3 of FIG. 3 described above. After extending expandable tip 201 as illustrated by FIG. 7, the user may pull back on plunger 213 until reaching a hard stop at stage 505. As a result of plunger 213 being pulled out, the volume of open space within housing 205 is increased, thereby creating a negative pressure within housing 205. This negative pressure causes extension tip 207 to at least partially collapse.

As illustrated by the upper image of FIG. 8, push rod 209 and extension tip 207 remain in the extended position after plunger 213 has been pulled back. This is due to the locking mechanism that holds push rod 209 in place once plunger 213 is moved to stage 503. Additionally, as described above, plunger 213 may not be connected to push rod 209 which allows plunger 213 to be pulled back without retracting push rod 209 and extension tip 207.

In another example, a detent is placed at stage 505 to prevent plunger 213 from sliding forward towards stage 503. The detent is directional, allowing guide pin 215 to move from stage 503 to 505, but preventing guide pin 215 from moving from stage 505 to stage 503. This may prevent, for example, the negative pressure within housing 205 from pulling plunger 213 forward, thereby re-inflating expandable tip 201.

In one example, when plunger 213 is rotated to stage 505, the front profile of expandable tip 201 may change. For example, FIG. 9 illustrates expandable tip 201 at stages 501 and 505/507. The top left image of FIG. 9 illustrates a front view of expandable tip 201 while plunger 213 is at stage 501. As shown in FIG. 9, the front profile of expandable tip 201 at stage 501 is substantially circular. In contrast, the bottom left image of FIG. 9 illustrates a front view of expandable tip 201 when plunger 213 is rotated to stages 505/507. In the illustrated example, the front profile of expandable tip 201 at stages 505/507 is both reduced in size and non-circular. By changing the shape and reducing the size of the front profile, the user is provided with an improved line of sight when inserting the device. In other words, the reduced profile allows the user to more easily view the target site and properly position collection and transfer device 100 for collecting the cell samples. In one example, the non-circular shape is generated by a non-uniform thickness of the sidewalls of expandable tip 201. For instance, certain portions of expandable tip 201 are given a greater thickness to decrease the amount that the sides are collapsed.

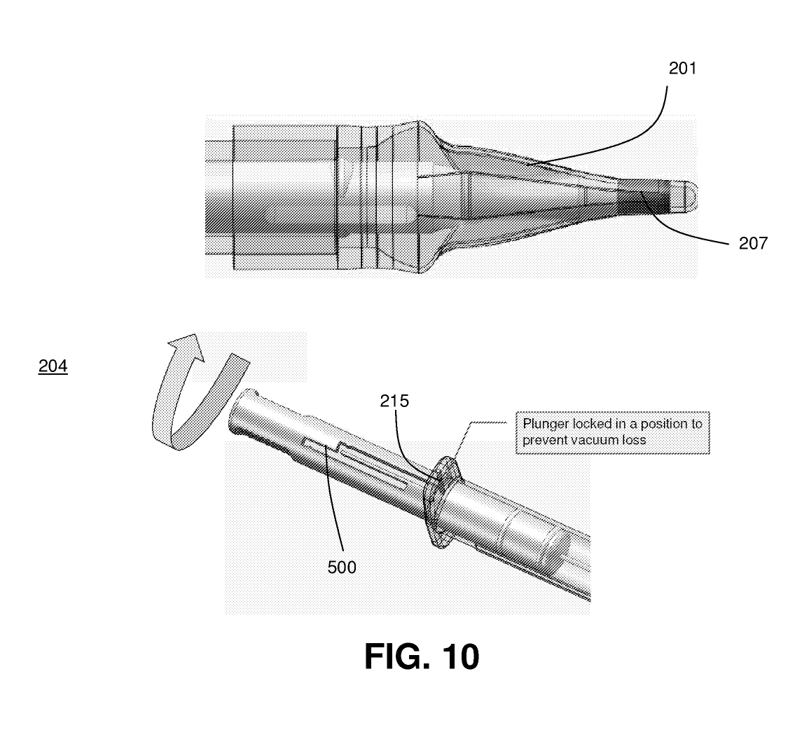

FIG. 10 illustrates an exemplary process for locking plunger 213 to prevent the plunger from being pulled back into housing 205 and causing vacuum loss. This process corresponds with block 4 of FIG. 3 described above. After expandable tip 201 is collapsed as illustrated by FIG. 8, the user may rotate plunger 213 counter-clockwise until guide pin 215 reaches stage 507. FIG. 10 shows a view of expandable tip 201 and corresponding view of main body 202 after the user has locked plunger 213.

In one example, after expandable tip 201 is collapsed and plunger 213 is locked in place, the user of the collection and transfer device 100 may insert the device into the patient's body. Using the improved line of sight provided by the reduced front profile of expandable tip 201, the user may position expandable tip 201 against the target site for collection of the cell samples. In another example, expandable tip 201 may not be able to make complete contact with the target site of the cervix. This may be due to substantial variation in the shape and dimensions of the cervix and cervical canal between women. To compensate for these variations, the user of collection and transfer device 100 may partially inflate expandable tip 201 to conform to the contours of the cervix. This may increase the surface area of expandable tip 201 in contact with the target site and allow collection and transfer device 100 to collect cell samples from a larger area of the cervix.

FIG. 11 illustrates an exemplary process for partially inflating expandable tip 201 while inside the patient's body. This process corresponds with block 5 of FIG. 3 described above. In one example, after expandable tip 201 has been positioned adjacent the target site, the user may push plunger 213 forward until reaching a hard stop at stage 509. As a result of plunger 213 being pushed in, expandable tip 201 is partially inflated due to the positive pressure created by plunger 213 within housing 205. In one embodiment, the diameter of expandable tip 201 increases from 2.5 mm to 6.5 mm (an increase of 160%), as between blocks 4 and 5 of FIG. 3, respectively. In other embodiments, the diameter of expandable tip 201 increases by 75%, 100%, 125%, 150%, 175%, or 200% as between blocks 4 and 5 of FIG. 3.

FIG. 11 shows a view of expandable tip 201 and a corresponding view of main body 204 after expandable tip 201 has been partially inflated.

In another example, a detent is placed at stage 509 to prevent plunger 213 from backtracking towards stage 507 and deflating expandable tip 201. The detent at stage 509 is directional, allowing movement of guide pin 215 from stage 507 towards stage 509 but preventing movement of guide pin 215 from stage 509 towards stage 507.

In one example, cell samples are collected on the portions of the surface of expandable tip 201 that is placed in contact with the target site. In one embodiment, collection involves twisting or rotating device 100 around its long axis by 20 degrees to facilitate additional cell collection. In other embodiments, device 100 may be rotated 5, 10, 15, 25, 30, or 35 degrees to facilitate transfer. After collection, the collection and transfer device 100 may be removed from the patient. The user may then fully inflate expandable tip 201 to prepare the tip for transferring the collected cells to a receiving surface. In one example, the receiving surface may be a slide, Petri dish, or the like. FIG. 12 illustrates an exemplary process for fully inflating expandable tip 201. This process corresponds with block 6 of FIG. 3 described above

In one example, after the cell samples are collected and the device is removed from the patient, the user may rotate plunger 213 counter-clockwise and push in plunger 213 until reaching stage 511 as illustrated by FIG. 12. As a result of plunger 213 being pushed in, expandable tip 201 is fully inflated due to the positive pressure within housing 205 created by plunger 213. In one example, a detent is placed at stage 511 to prevent plunger 213 from moving towards stage 513 and deflating expandable tip 201. The detent at stage 511 is directional, allowing movement of guide pin 215 towards stage 511 but restricting the movement of guide pin 215 from stage 511 towards stage 513.

Once expandable tip 201 is fully inflated, expandable tip 201 is ready to transfer the cells contained in the surface of the tip to a receiving surface. In one example, this is accomplished by putting the surface of expandable tip 201 in contact with the receiving surface. The user may press the end of expandable tip 201 down onto the receiving surface to stamp the cells onto the receiving surface. By transferring the cells in this manner, the spatial relationship between the cells is maintained from the target site, to the expandable tip, and finally to the receiving surface.

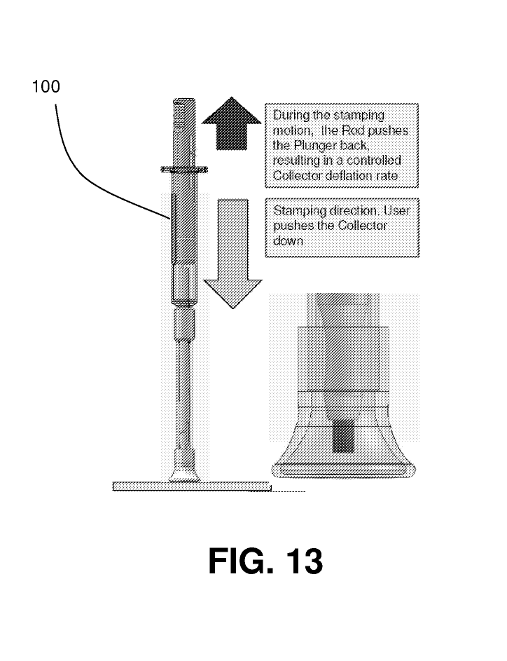

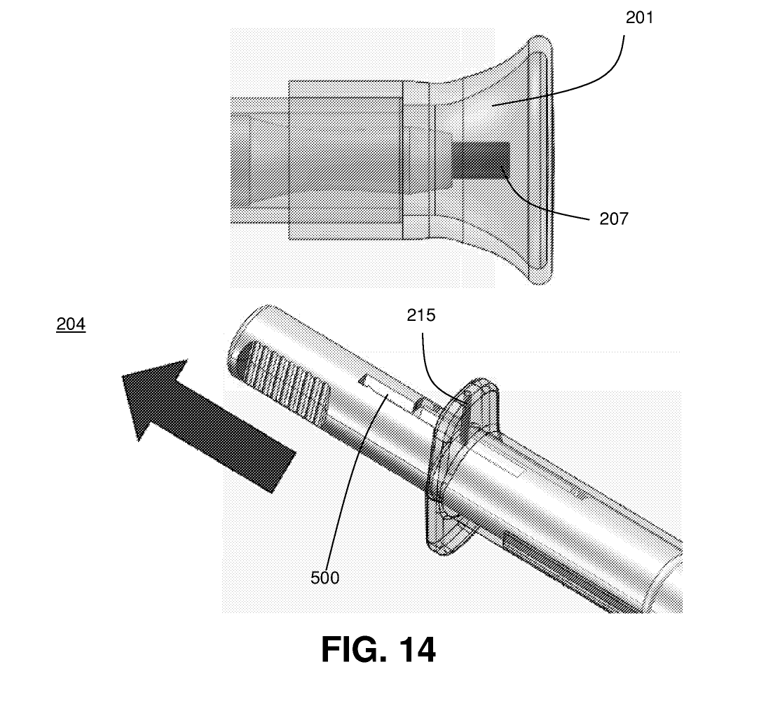

In another example, illustrated by FIGS. 13 and 14 and corresponding with block 7 of FIG. 3 described above, when expandable tip 201 is pressed against the receiving surface, pressure is applied to extension tip 207 and push rod 209. This pressure causes a release of the lock on push rod 209 that was activated at stage 503. Once the lock is released, the pressure applied to extension tip 207 and push rod 209 causes plunger 213 to slide backwards and slowly deflate expandable tip 201. Alternatively, a valve (not pictured) may be used to generate a controlled release of the pressure within housing 205, thereby deflating expandable tip 201. In one embodiment, the valve is a friction controlled opening or orifice that is exposed by the pressure applied to extension tip 207. As illustrated by FIGS. 13 and 14, the deflation allows expandable tip 201 to conform to the receiving surface to more accurately transfer the cell samples. In one example, where the locking mechanism is a detent, the lock is released by applying a sufficient amount of pressure to force push rod 209 past the detent. In another example, where the locking mechanism is a rotational lock, the user is required to rotate push rod 209 out of a locked position in order to retract push rod 209.

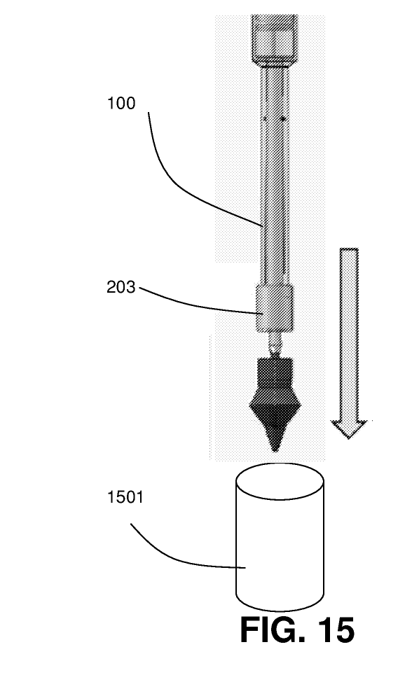

In another example illustrated by FIG. 15, a user may remove expandable tip 201 from main body 204 for later processing. This process corresponds with block 7 of FIG. 3 described above. In this example, expandable tip 201 is removed from main body 204 instead of stamping the cells onto a receiving surface. In another example, expandable tip 201 is removed from main body 204 after stamping the cells onto the receiving surface. The user may remove expandable tip 201 by sliding stripper ring 203 forward until making contact with expandable tip 201. The force applied to stripper ring 203 and subsequently to expandable tip 201 separates the tip from main body 204 without the user having to make contact with and contaminating expandable tip 201. In one example, expandable tip 201 is removed using stripper ring 203 and placed in vial 1501.

In one example, the receiving surface may be a specially treated slide. For example, the slide may be treated with a chemical to allow a greater number of cell samples to adhere to the slide. Such chemicals are known to those of ordinary skill in the art and any such chemical may be used.

In another example, the receiving surface may be treated with a fixative chemical after the cell samples have been transferred. The fixative chemical may be used to prevent the cell samples from sliding while on the receiving surface. Such fixative chemicals are known to those of ordinary skill in the art and any such chemical may be used.

What is claimed is:

1. An apparatus for collecting and transferring cell samples, the apparatus comprising:
a main body;
an expandable tip coupled to the main body and configured to:
inflate to a first inflation level for collecting a cell sample,
inflate to a second inflation level for transferring at least a portion of the collected cell sample, and
deflate, at a rate of deflation, to a deflated level when the at least a portion of the collected cell sample is being transferred;
a valve, for controlling the rate of deflation of the expandable tip; and
a plunger disposed within the main body configured to generate positive pressure in the expandable tip.

2. The apparatus of claim 1, further comprising:
a guide-pin connected to the main body;
at least one slot along a surface of the plunger, wherein the guide-pin is positioned within the at least one slot to guide the plunger to a plurality of positions within the main body; and
at least one directional detent to restrict movement of the plunger within the main body.

3. The apparatus of claim 1, further comprising a push rod coupled to the plunger.

4. The apparatus of claim 3, wherein the plunger is operable to extend the expandable tip using the push rod.

5. The apparatus of claim 3, further comprising a pressure release lock connected to the main body, wherein the pressure release lock is operable to restrict movement of the push rod relative to the main body, and wherein the pressure release lock is operable to allow the push rod to slide relative to the main body to deflate the expandable tip when pressure is applied to a tip connected to the push rod.

6. The apparatus of claim 1, wherein the plunger is operable to pneumatically collapse the expandable tip into a collapsed state, and wherein a front profile of the expandable tip in the collapsed state is smaller than a front profile of the expandable tip in a non-collapsed state.

7. The apparatus of claim 1, wherein the plunger is operable to inflate the expandable tip to the first inflation level.

8. The apparatus of claim 1, wherein the plunger is operable to inflate the expandable tip to the second inflation level.

9. The apparatus of claim 1, wherein the expandable tip is made of an elastic polymer.

10. The apparatus of claim 1, wherein the elastic polymer is Dynaflex™ G6713-0001.

11. The apparatus of claim 1, further comprising:
a stripper ring coupled to the main body for removing the expandable tip.

12. The apparatus of claim 1, wherein the diameter of the expandable tip at the first inflation level is 100%-200% larger than the diameter of the expandable tip in an un-inflated state.

13. The apparatus of claim 1, wherein the expandable tip is operable to collect a cell sample comprising at least 55,000 cells.

14. The apparatus of claim 13, wherein the expandable tip is further operable to transfer at least 50,000 cells of the cell sample to a receiving surface.

15. The apparatus of claim 1, wherein the expandable tip is operable to collect the cell sample in response to a 10-20 degree rotation of the apparatus while the expandable tip is in contact with a target site.

16. An apparatus for collecting and transferring cell samples, the apparatus comprising:
a main body;
an expandable tip coupled to the main body and configured to:
inflate to a first inflation level for collecting a cell sample,
inflate to a second inflation level for transferring at least a portion of the collected cell sample, and
deflate, at a rate of deflation, to a deflated level when the cell sample is being transferred;
a plunger disposed within the main body configured to generate positive pressure in the expandable tip, wherein the plunger is further configured to deflate the expandable tip.

17. A method of using an apparatus for collecting and transferring cells, the method comprising:
creating a vacuum to collapse a tip of the apparatus to allow for visualization of a target site;
inserting the apparatus into a patient and contacting the target site with the tip of the apparatus;
inflating the tip to expand the tip;
while the apparatus is in contact with the target site, rotating the apparatus to collect a cell sample;
removing the apparatus from the patient;
further inflating the tip to further expand the tip;
transferring the at least a portion of the cell sample to a receiving surface by applying pressure to the tip, wherein the tip deflates in response to the applied pressure at a controlled rate.

18. The method of claim 17, wherein the at least a portion of the cell sample transferred includes fewer cells than the cell sample, and wherein the method further comprises:
after transferring the at least a portion of the cell sample to the receiving surface, reinflating the tip;
transferring a second portion of the cell sample to a second receiving surface by applying a second pressure to the tip, wherein the tip deflates in response to the applied second pressure.

19. The method of claim 17, further comprising treating the receiving surface with a chemical prior to transferring the at least a portion of the cell sample to the receiving surface.

20. The method of claim 1, further comprising treating the receiving surface with a chemical after transferring the at least a portion of the cell sample to the receiving surface.
